SEQUENCE LISTING (5 sequences)

SEQ ID NO: 1 - protein, 456 aa, Bos sp.
TNPGIVARITQKGLDYACQQGVLTLQKELEKITIPNFSGNFKIKYLGKGQYSFFSMVIQGFNLPNSQIRPLPDKGLDLSIRDASIKIRGKWKARKNFIKLGGNFDLSVEGISILAGLNLGYDPASGHSTVTCSSCSSGINTVRIHISGSSLGWLIQLFRKRIESLLQKSMTRKICEVVTSTVSSKLQPYFQTLPVTTKLDKVAGVDYSLVAPPRATANNLDWLLKGEFFSLAHRSPPPFAPPALAFPSDHDRMVYLGISEYFFNTAGFVYQKAGALNLTLRDDMIPKESKFRLTTKFFGILIPQVAKMFPDMQMQLFIWASLPPKLTMKPSSLDLIFVLDTQAFAILPNSSLDPLFLLEMNLNLSVVVGAKSDRLIGELRLDKLLLELKHSDIGPFSVESLQSVINYVMPTIVLPVINKKLQKGFPLPLPAYIELFNLTLQPYQDFLLFGADVQYS

SEQ ID NO: 2 - protein, 35 aa, Bos sp.
NSQIRPLPDKGLDLSIRDASIKIRGKWKARKNFIK

SEQ ID NO: 3 - protein, 28 aa, Bos sp.
VRIHISGSSLGWLIQLFRKRIESLLQKS

SEQ ID NO: 4 - protein, 24 aa, Bos sp.
KWKARKNFIKGSSLGWLIQLFRKR

SEQ ID NO: 5 - protein, 24 aa, Bos sp.
MCHWAGGASNTGDARGDVFGKQAG

(19) United States
(12) Patent Application Publication
(10) Pub. No.: US 2009/0048160 A1
(43) Pub. Date: Feb. 19, 2009

(54) Antimicrobial activity of bovine bactericidal/permeability-increasing protein (BPI)-derived peptides against Gram-negative bacterial mastitis isolates

(76) Inventors: Douglas D. Bannerman, Baltimore, MD (US); Dante S. Zarlenga, Ellicott City, MD (US); Annapoorani Chockalingam, Ithaca, NY (US)

Correspondence Address: USDA, ARS, OTT, 5601 SUNNYSIDE AVE, RM 4-1159, BELTSVILLE, MD, 20705-5131, US

(21) Appl. No.: 11/893,964

(22) Filed: Aug. 17, 2007

(51) Int. Cl. C07K 14/00; A01P 1/00; C12N 1/00; C12N 15/63; C12N 15/11; A61K 38/16

(52) U.S. Cl. 514/12; 435/243; 435/320.1; 530/324; 530/350; 536/23.1

(57) ABSTRACT

The antimicrobial activity of bovine bactericidal/permeability-increasing protein (bBPI)-derived synthetic peptides against mastitis-causing Gram-negative bacteria was evaluated. Three peptides were synthesized with sequences corresponding to amino acids 65-99 (bBPI₆₅₋₉₉), 142-169 (bBPI₁₄₂₋₁₆₉), or the combination of amino acids 90-99 and 148-161 (bBPI_{90-99,148-161}) of bBPI. The bBPI_{90-99,148-161} peptide demonstrated the widest spectrum of antimicrobial activity, with minimum inhibitory (MIC) and bactericidal (MBC) concentration values ranging from 16-64 μg/ml against Escherichia coli, Klebsiella pneumoniae, and Enterobacter spp, and 64-128 μg/ml against Pseudomonas aeruginosa. None of the peptides exhibited any growth inhibitory effect on Serratia marcescens. The antimicrobial activity of bBPI_{90-99,148-161} was inhibited in milk, but preserved in serum. Finally, both bBPI₁₄₂₋₁₆₉ and bBPI_{90-99,148-161} were demonstrated to completely neutralize LPS. The peptide bBPI_{90-99,148-161} is a potent neutralizer of the highly pro-inflammatory molecule bacterial LPS and has antimicrobial activity against a variety of Gram-negative bacteria.

### BACKGROUND OF THE INVENTION

1. Field of the Invention

This invention relates to the antimicrobial activity of bovine bactericidal/permeability-increasing protein (BPI)-derived synthetic peptides against mastitis-causing Gram-negative bacteria. A preferred embodiment relates to three synthetic peptides with sequences corresponding to amino acid sequences of bovine BPI (bBPI), namely, amino acids 65-99 (bBPI₆₅₋₉₉), 142-169 (bBPI₁₄₂₋₁₆₉), or the combination of amino acids 90-99 and 148-161 (bBPI_{90-99,148-161}) of bBPI. The invention further relates to therapeutic application for these peptides in treating diseases of food production animals caused by Gram-negative bacteria, for example, mastitis and septicemia, and in managing complications associated with such diseases, including septic shock.

2. Description of the Relevant Art

Mastitis continues to be among the most costly diseases to the dairy industry and annual economic losses attributed to this disease in the United States are estimated to approach two billion dollars (Wells et al. 1998. J. Dairy Sci. 81: 3029-3035). Worldwide, mastitis is associated with economic losses of 35 billion dollars annually (Wellenberg et al. 2002. Vet Microbiol. 88: 27-45). Decreased milk production and quality, as well as veterinary expenses, all contribute to these economic losses. Further, because mastitis is among the leading reasons for culling cows, animal replacement costs contribute to the financial burden that this disease imposes on producers (Bascom and Young. 1998. J. Dairy Sci. 81: 2299-2301).

From an animal health perspective, mastitis is one of the most frequent diseases of dairy cows and a leading cause of death and culling (Lescourret and Coulon. 1994. J. Dairy Sci. 77: 2289-2301; Seegers et al. 2003. Vet. Res. 34: 475-491; Esslemont and Kossaibati. 1997. Vet. Res. 140: 36-39). Although implementation of preventative measures, including pre- and post-teat disinfection, have been effective at reducing intramammary infection caused by contagious pathogens, these measures have had relatively little impact on controlling mastitis caused by environmental pathogens (DeGraves and Fetrow. 1993. Vet. Clin. North Am. Food Anim. Pract. 9: 421-434; Hillerton et al. 1995. J. Dairy Res. 62: 39-50; Oliver et al. 1990. J. Dairy Sci. 73: 2230-2235). As a result, the proportion of cases of mastitis due to environmental bacteria has increased.

Gram-negative bacteria are among the most common environmental pathogens to cause mastitis and are responsible for approximately one-third of all clinical cases of bovine mastitis (Anderson et al. 1982. J. Am. Vet. Med. Assoc. 181: 690-693; Hogan et al. 1989. J. Dairy Sci. 72: 1547-1556). Nearly 25% of the most severe cases result in culling or death of the animal (Eberhart, R. J. 1984. Vet. Clin. North Am. Food Anim. Pract. 6: 287-300). Of the Gram-negative bacteria that cause bovine mastitis, Escherichia coli are the most prevalent (Makovec and Ruegg. 2003. J. Dairy Sci. 86: 3466-3472; Wilson et al. 1997. J. Dairy Sci. 80: 2592-2598). Other common Gram-negative isolates include bacteria from the Klebsiella, Serratia, Enterobacter, and Pseudomonas genera. An inverse relationship between the incidence of Gram-negative infections and bulk tank milk somatic cell counts has been reported (Barkema et al. 1998. J. Dairy Sci. 81: 411-419), thus, the cases of mastitis caused by these infections are expected to increase as dairymen continue to strive for lower milk somatic cell counts. Unfortunately, current antibiotic therapy for the treatment of intramammary Gram-negative bacterial infections remains suboptimal.

It has been estimated that 10-30% of the clinical cases of mastitis caused by Gram-negative pathogens develop into severe peracute mastitis (Erskine et al. 1991. J. Am. Vet. Med. Assoc., 198: 980-984; Ziv, G. 1992. Vet. Clin. North Am. Food Anim. Pract. 8: 1-15) and bacteremia is associated with a significant number of these cases (Cebra et al. 1996. J. Vet. Intern. Med. 10:252-257; Wenz et al. 2001. J. Am. Vet. Med. Assoc. 219: 976-981). The systemic complications and deleterious outcome associated with Gram-negative infection is the result of an exaggerated inflammatory response elicited largely by a highly pro-inflammatory component of the Gram-negative bacterial envelope known as endotoxin or bacterial lipopolysaccharide (LPS) (Erskine et al., supra; Ziv, supra; Jackson and Bramley. 1983. In Pract. 5: 135-146). The bovine mammary gland is highly sensitive to low doses of LPS (Carroll et al. 1964. Am. J. Vet. Res. 25: 720-726; Mattila and Frost. 1989. Res. Vet. Sci. 46: 238-240). Injection of LPS into the mammary glands of healthy cows induces mastitis (Carroll et al., supra; Paape et al. 1974. Proc. Soc. Exp. Biol. Med. 145: 553-559; Anri, A. 1989. Nippon Juigaku Zasshi 51: 847-848) and LPS is detectable in the milk of cows with coliform mastitis (Anri, supra; Hakogi et al. 1989. Vet. Microbiol. 20: 267-274). Absorption of LPS into blood following its injection into healthy mammary glands (Ziv et al. 1976. Theriogenology 6: 343-352) and during naturally occurring E. coli mastitis (Katholm and Andersen. 1992. Vet. Rec. 131: 513-514) has been reported. In those cases where bacteremia develops, LPS is introduced directly into the circulation. It is well-established that the systemic inflammatory response that accompanies peracute coliform mastitis is mediated, in part, by LPS (Ziv, supra; Jackson and Bramley, supra; Carroll et al., supra; Eberhart et al. 1979. J. Dairy Sci. 62: 1-22; Schalm et al. 1964. Am. J. Vet. Res. 25: 75-82); however, therapeutic treatment to counteract the excessive inflammatory response elicited by LPS remains lacking. Conventional antimicrobials that are approved for internal (i.e., non-topical) use target the pathogen and do not target the LPS molecule. By targeting the pathogen, conventional antimicrobials can cause an increase in the circulating levels of LPS by inducing bacterial death and corresponding LPS shedding, thereby, exacerbating the deleterious inflammatory response (Morrison and Bucklin. 1996. Scand. J. Infect. Dis. Suppl. 101: 3-8). In addition to mastitis, Gram-negative bacteria are responsible for several other economically important diseases of food production animals, i.e., cattle, sheep, goats, and pigs, including: enteric colibacillosis, coliform septicemia, brucellosis, metritis, salmonellosis, and campylobacteriosis. In fact, most of the clinical infections and mortality associated with food-animal neonates result from Gram-negative organisms and the ensuing host inflammatory responses (Cullor, J. S. 1992. J. Am. Vet. Med. Assoc. 200: 1894-1902). Therefore, development of novel interventions that can moderate the inflammatory responses elicited by LPS remains an important animal health goal.

Synthetic congeners corresponding to the functional domains of naturally-occurring endogenous antimicrobial and/or LPS-neutralizing proteins can aid in managing Gram-negative infections in cattle and other agriculturally-relevant animals. Bactericidal/Permeability-increasing Protein (BPI) is a 55 kDa cationic protein expressed in the primary (azurophilic) granules of human neutrophils, where it constitutes nearly 0.5-1% of the total cellular protein content (Elsbach, P. 1998. J. Leukoc. Biol. 64: 14-18). BPI possesses bactericidal, LPS-binding, and opsonic activity, all of which contribute to its role in host defense. In a variety of experimental animal models and human studies, recombinant human BPI was therapeutically efficacious in the treatment of Gram-negative infections and the attendant complications associated with such infections (Kelly et al. 1993. Surgery 114:140-146; Kohn et al., 1993. J. Infect. Dis. 168:1307-1310; Levin et al., 2000. Lancet356: 961-967; Lin et al. 1996. Antimicrob. Agents Chemother. 40: 65-69; Rogy et al., 1994. J. Clin. Immunol. 14:120-133).

Recombinant human BPI is therapeutically efficacious in the treatment of Gram-negative infections. There have been reports establishing that homologous sequences of functional domains within the human ortholog possess antimicrobial and/or LPS-neutralizing activity. The cDNA sequence of the mature bBPI protein is moderately conserved at the nucleotide (75%) and amino acid (63%) levels when compared with human BPI (Leong and Camerato. 1990. Nucleic Acids Res. 18: 3052). The amino-terminal region spanning amino acids 1-199 of human BPI contains the functional domains that confer both bactericidal and LPS-neutralizing activity (Gazzano-Santoro et al. 1992. Infect. Immun. 60: 4754-4761). These activities are governed by short regions within the amino-terminus, including regions corresponding to amino acids 65-99 and 142-169 (Gray and Haseman. 1994. Infect. Immun. 62: 2732-2739; Little et al. 1994. J. Biol. Chem. 269: 1865-1872). Within amino acids 65-99 of human BPI, bactericidal activity is conferred by a subset region within amino acids 85-99. A synthetic peptide corresponding to amino acids 90-99 of human BPI possesses bactericidal, but not LPS-neutralizing activity (Gray and Haseman, supra) whereas a peptide corresponding to amino acids 148-161 of human BPI neutralizes LPS (Jiang et al. 2004. Int. Immunopharmacol. 4: 527-537). Peptides composed of fused sequences of amino acid regions from different functional domains within human BPI confer both bactericidal and LPS-neutralizing properties (Gray and Haseman, supra; Little, R. G., U.S. Pat. No. 6,495,516. Dec. 17, 2002 and Little et al., U.S. Pat. No. 7,045,500. May 16, 2006).

In contrast to human BPI, little is known about the functional activity of bBPI or bovine peptides that are homologous to regions governing functional activity in the human ortholog. Because of the documented involvement of Gram-negative bacteria in a large portion of clinical cases of bovine mastitis and other diseases of cattle, there is a need to determine the bactericidal and LPS-neutralizing activity of synthetic peptides of bBPI.

### SUMMARY OF THE INVENTION

We have discovered that three synthetic peptides with amino acid sequences corresponding to sequences of bBPI, namely, amino acids 65-99 (bBPI₆₅₋₉₉), 142-169 (bBPI₁₄₂₋₁₆₉), and the combination of amino acids 90-99 and 148-161 of bBPI (bBPI_{90-99,148-161}) exhibit antimicrobial activity and/or LPS neutralizing activity against mastitis-causing Gram-negative bacteria.

In accordance with this discovery, it is an object of the invention to provide an antimicrobial peptide, corresponding to the combination of amino acids 90-99 and 148-161 (bBPI_{90-99,148-161}) of bBPI, which exhibits antimicrobial activity and LPS neutralizing activity against mastitis-causing Gram-negative bacteria.

It is also an object of the invention to provide the peptides, corresponding to amino acids 65-99 (bBPI₆₅₋₉₉) and 142-169 (bBPI₁₄₂₋₁₆₉), of bBPI, which exhibit LPS neutralizing activity against mastitis-causing Gram-negative bacteria.

It is an additional object of the invention to provide novel nucleic acid molecules which encode the amino acids 65-99 (bBPI₆₅₋₉₉), 142-169 (bBPI₁₄₂₋₁₆₉), or the combination of amino acids 90-99 and 148-161 (bBPI_{90-99,148-161}) of bBPI.

An added object of the invention is to provide peptides which are therapeutic for food production animals. It is well understood that these sequences can also be used in combination with another sequence, or sequences, exhibiting one or more anti-mastitis therapeutic properties.

A further object of the invention is to provide pharmaceutical compositions useful for the treatment of diseases caused by Gram-negative bacteria.

An additional object of the invention is to provide pharmaceutical compositions useful for the management of Gram-negative induced mastitis and septicemia and complications caused such diseases, such as septic shock.

Also part of this invention is a kit, comprising a pharmaceutical composition for treatment of disease caused by Gram-negative bacteria.

Other objects and advantages of this invention will become readily apparent from the ensuing description.

### BRIEF DESCRIPTION OF THE DRAWINGS

FIG. 1 depicts the mean (±S.E.) percent neutralization of bacterial LPS bioactivity as determined with the Limulus amebocyte lysate assay. Increasing concentrations of peptides or polymixin B were incubated with 1 ng of E. coli-derived LPS for 30 min. Following incubation, the amount of free LPS was determined by extrapolation from a standard curve and the percent of bound (neutralized) LPS was calculated.

### DETAILED DESCRIPTION OF THE INVENTION

Gram-negative bacteria are involved in a large portion of clinical cases of bovine mastitis. This invention relates to the design and creation of new antimicrobials. Little is known about the functional activity of bBPI or peptides corresponding to sequences within this protein that are homologous to regions governing antimicrobial activity in the human ortholog. Here, we have evaluated the bacteriostatic, bactericidal, and LPS-neutralizing activity of peptides corresponding to specific amino acid sequences within bBPI. Two peptides were synthesized whose sequence corresponded to amino acids 65-99 and 142-169 of the mature bBPI protein (SEQ ID NO:1). A third fusion peptide corresponding to bBPI amino acids 90-99 and 148-161 was evaluated to determine whether the fusion of sequences of amino acid regions from two hypothesized functional domains of bBPI could confer bifunctionality, i.e., bactericidal and LPS-neutralizing properties. The bBPI peptides are homologous to regions involved in antimicrobial activity in the human BPI ortholog, but differ in amino acid sequences and in antimicrobial activities associated with the peptides. For example, while the human homolog of the bBPI₆₅₋₉₉ peptide is bactericidal, bBPI₆₅₋₉₉ does not exhibit bactericidal activity (see Example 3, Table 1).

Three bBPI peptides, bBPI₆₅₋₉₉, bBPI₁₄₂₋₁₆₉, and bBPI_{90-99,148-161}, were evaluated for growth-inhibitory and bactericidal activities against Gram-negative bacteria isolates from clinical cases of mastitis. Of the three peptides tested, bBPI_{90-99,148-161}, had the lowest minimum inhibitory concentration (MIC) and minimum bactericidal concentration (MBC) values against the widest spectrum of clinical isolates (See Example 3, Table 1). E. coli, K. pneumoniae, and Enterobacter spp. were the pathogens most sensitive to the bacteriostatic and bactericidal effects of the peptide, whereas, P. aeruginosa displayed an intermediate sensibility. All isolates of S. marcescens were completely resistant to the growth-inhibitory and bactericidal activities of this peptide. Because others have similarly reported that S. marcescens is resistant to the bactericidal effects of human and rabbit BPI or their derivatives (Little, R. G. 2002, supra, Beckerdite et al. 1974. J. Exp. Med. 140: 396-409; Levy et al. 2000. Infect. Immun. 68: 5120-5125), it is not surprising that this bacterium may be resistant to bBPI, and thus, its peptide derivatives. S. marcescens resistance to a variety of antibiotics, including β-lactams, aminoglycosides, and quinolones, has been reported (Acar et al. 1993. Drugs: 45 Suppl 3: 24-28; Fish et al., 1995. Pharmacotherapy 15: 279-291; Jones, R. N. 1992. Diagn. Microbiol. Infect. Dis. 15: 3S-10S). In addition, S. marcescens displays resistance to common disinfectants, including chlorhexidine which is used in some teat dips (Hogan et al. 1995. J. Dairy Sci. 78: 2502-2506; Marrie et al. 1981. Appl. Envrion. Microbiol. 42: 1093-1102). Mechanisms for resistance to these antibiotics include: (1) production of enzymes that destroy the antibiotic (Farar and O'Dell. 1976. J. Infect. Dis. 134: 245-251; Hechler et al. 1989. J. Gen Microbiol. 135: 1275-1290); (2) expression of efflux pumps that decrease accumulation of antibiotics within the cell (Berlanga et al. 2000. Microb. Drug. Resist. 6: 111-117; Kumar and Worobec. 2005. Antimicrob. Agents Chemother. 49: 1495-1501); and (3) alteration of porin expression, which reduces membrane permeability to antibiotics (Hashizume et al. 1993. J. Antimicrob. Chemother. 31: 21-28; Ruiz et al. 2003. Microb. Drug Resist. 9: 257-264). Whether these effector mechanisms of S. marcescens confer resistance to the antimicrobial effects of BPI or its peptide derivatives remains unknown.

For those bacteria that were sensitive to bBPI_{90-99,148-161}, the MIC values of the peptide tended to be higher than those of tetracycline (see Example 3, Table 1). The MBC values of the peptide tended to be lower than those of tetracycline as was the MBC:MIC ratio, consistent with the respective bactericidal and bacteriostatic actions of BPI and tetracycline. In accordance with quality control guidelines (NCCLS, 1999), two reference strains of bacteria, E. coli and S. aureus, were evaluated and the tetracycline MIC values were within established quality control ranges. Interestingly, the S. aureus reference strain exhibited sensitivity to the bactericidal effects of bBPI₁₄₂₋₁₆₉ and bBPI_{90-99,148-161}. Although S. aureus is reportedly resistant to the bactericidal activity of the full-length human BPI protein, peptides corresponding to either amino acids 90-99 of this protein or the combination of regions 90-99 and 148-161 effectively kill S. aureus at concentrations comparable to those that are bactericidal against E. coli (Gray and Haseman, supra; Little, 2002, supra; Hovde and Gray. 1986. Infect Immun. 54: 142-148). Thus, the finding that bBPI_{90-99,148-161} exhibits bactericidal activity against S. aureus is consistent with the results from studies of peptides derived from human BPI.

Despite findings that the bactericidal activity of human BPI can be localized to amino acids 65-99 in the native protein and that synthetic peptides corresponding to amino acids 90-99 of the human protein are bactericidal (Gray and Haseman, supra; Little et al., supra), as discussed above, bBPI₆₅₋₉₉ exhibits a complete lack of bactericidal activity against any of the bacterial isolates tested (see Example 3, Table 1), thus underscoring the functional differences between the human and bovine homologs. One explanation may be the differences in amino acid composition between the human and bBPI within this region of each protein. However, while bBP₆₅₋₉₉ lacked activity, bBPI_{90-99,148-161} demonstrated bactericidal activity against a wide-spectrum of isolates. That the other peptide tested, bBPI₁₄₂₋₁₆₉, showed relatively modest antimicrobial activity against a limited number of isolates and at MIC's and MBC's higher than bBPI_{90-99,148-161}, suggests that the bactericidal activity of bBPI_{90-99,148-161} is largely governed by amino acids 90-99 of bBPI. Because these amino acids are contained within bBPI₆₅₋₉₉, it is surprising that this peptide did not have similar antimicrobial activity to that of bBPI_{90-99,148-161}. This finding suggests that amino acids N- or C-terminal to amino acids with functional activity can affect the properties governed by that sequence. Further evidence supporting this contention comes from studies with human BPI and includes: 1) addition of a single cysteine amino acid to the amino-terminus of a human BPI synthetic peptide enhances bactericidal activity by approximately 10-fold; and 2) peptides corresponding to sequences within human BPI, but not the whole protein, exert bactericidal activity against P. aeruginosa (Gray and Haseman, supra). In a study with another peptide corresponding to the C-terminus of the protein chimerin, addition of a single amino acid reduced activity by several orders of magnitude (Wittamer et al. 2004. J. Biol. Chem. 279: 9956-9962). Thus, the amino acids N-terminal to the region corresponding to amino acids 90-99 of bBPI₆₅₋₉₉ may impair the activity governed by this region and ablate corresponding bactericidal activity relative to that observed for the bBPI_{90-99,148-161} peptide.

Because constituents of physiological fluids can inhibit the activity of various antimicrobial compounds (Miles and Maskell. 1986. J. Antimicrob. Chemother. 17: 481-487; Miles et al. 1986. J. Antimicrob. Chemother. 18: 185-193), the ability of bBPI_{90-99,158-161} to retain its activity in serum and milk was evaluated. The bactericidal activity of this peptide in these fluids was evaluated against the E. coli P4 strain, which was isolated from a natural case of clinical bovine mastitis and whose pathogenesis has been well-characterized (Bramley, supra; Bannerman et al. 2004. Clin. Diagn. Lab. Immunol. 11: 463-472; Anderson et al. 1977. Vet. Pathol. 14: 618-628) The bBPI_{90-99,148-161} peptide retained bactericidal activity against the E. coli P4 strain in serum (see Example 4, Table 2), but not in milk (see Example 4, Table 3). In whey, which is the protein-rich liquid fraction of milk devoid of cells, casein, and fat, the peptide exhibited bactericidal activity that was equivalent to that in serum (see Example 4, Table 4). Cations, including magnesium and calcium, have been reported to impair the activity of or sensitivity to an array of antibiotics (Miles and Maskell, supra; Miles et al., supra; Russell, A. D. 1967. J. Appl. Bacteriol. 30:395-401) as well as human and rabbit BPI (Weiss et al. 1978. J. Biol. Chem. 253: 2664-2672; Weiss et al. 1975. J. Clin. Invest. 55: 33-42) Because calcium and magnesium are present in bovine milk at ˜11- and 3-fold higher concentrations than in blood, respectively (Schalm et al. 1971. In Bovine Mastitis, Lea & Febigier Co., Philadelphia, Pa.), the elevated levels of one or both of these cations may be responsible for the inability of the peptide to exert its bactericidal effect in milk. In bovine milk, ˜25% and 65% of total magnesium and calcium, respectively, are associated with casein (Gaucheron, F. 2005. Reprod. Nutr. Dev. 45: 473-483; Neville, M. (C. 2005. J. Mammary Gland Biol. Neoplasia 10: 119-128; Fransson et al. 1983. Pediatr. Res. 17: 912-915). Thus, the finding that the BPI peptide retained bactericidal activity in whey, which is depleted of casein and contains correspondingly reduced levels of calcium and magnesium compared with milk, is consistent with the hypothesized impairment of the peptide's activity by the presence of these cations at elevated concentrations. These data, however, do not rule out the possibility that other constituents of milk contribute to or are solely responsible for the impairment bBPI_{90-99,148-161} bactericidal activity.

The finding that bBPI_{90-99,148-161} retains antimicrobial activity in serum suggests the therapeutic potential of this peptide in the treatment of septicemia. Pharmaceutical compositions comprising this peptide can be administered intravenously for the treatment of septicemia. While the lack of bactericidal activity of the peptide in milk may appear to negate its promise as a therapeutic, it is important to note that during experimentally-induced and naturally-occurring mastitis, the levels of calcium and magnesium in milk decrease by >50% (Bogin and Ziv. 1973. Cornell Vet. 63: 666-676; El Zubeir et al. 2005. J. S. Afr. Vet. Assoc. 76: 22-25). Thus, alterations in the composition of milk during the course of mastitis may enable bBPI_{90-99,148-161} to exert its bactericidal effect. Treatment of intramammary Gram-negative infections routinely occurs in response to the development of severe clinical signs, a time that often coincides with the greatest change in milk composition. Therefore, it is during this critical time of intervention when BPI may be able to exert a therapeutic effect when infused into the mammary gland. Thus, pharmaceutical compositions comprising the bBPI_{90-99,148-161} peptide can be administered intravenously and intramuscularly.

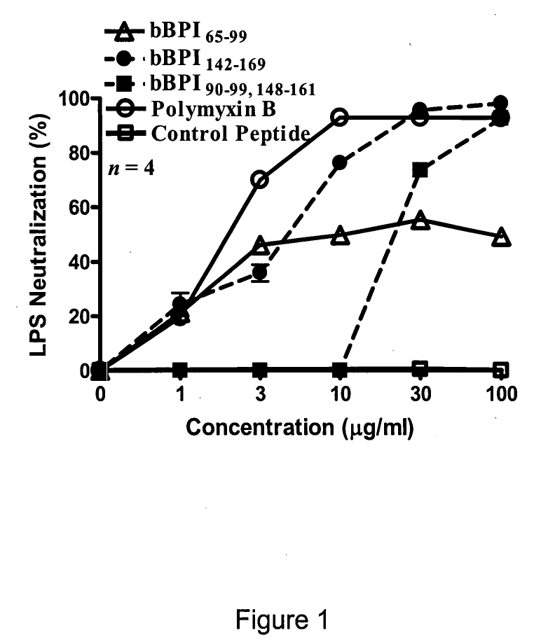

In addition to bactericidal activity, human BPI and its peptide derivatives bind to and neutralize LPS (Little, supra; Jiang et al., supra; Marra et al. 1990. J. Immunol. 144: 662-666). Peptide domain mapping has identified several regions within human BPI, including amino acids 65-103 and 137-171, which are associated with inhibition of LPS bioactivity. Synthetic peptides corresponding to sequences within these regions neutralize LPS in the Limulus amebocyte lysate (LAL) assay (Little, supra; Jiang et al., supra). Consistent with homologous human peptides, all three bBPI peptides, although differing from human homologs in their amino acid sequence, exhibited a partial ability to neutralize LPS (see FIG. 1). As determined by the lowest peptide concentration at which LPS was completely neutralized, bBPI₁₄₂₋₁₆₉ was the most effective of the peptides. bBPI_{90-99,148-161} inhibited 74% of LPS bioactivity at a peptide concentration of 30 μg/ml, whereas at 100 μg/ml, the inhibition was nearly complete. In terms of bifunctional capability to both kill bacteria and neutralize LPS, bBPI_{90-99,148-161} was the only peptide to exhibit both effects at concentrations <100 μg/ml. The enhanced ability of polymyxin B to neutralize LPS relative to bBPI₁₄₂₋₁₆₉ is consistent with findings comparing polymyxin B and a peptide corresponding to amino acids 148-161 of human BPI (Jiang et al., supra).

Results from the current study evaluating peptides corresponding to regions within this protein suggest that endogenous bBPI contains both LPS-neutralizing and bactericidal properties similar to its human and rabbit orthologs. The combined bactericidal and LPS-neutralizing activity of BPI and its derivatives indicates their therapeutic applicability to the treatment of Gram-negative infections (Levy, O. 2002. Expert. Opin. Investig. Drugs 11: 159-167). The life-threatening systemic complications that arise in response to these infections are due, in part, to the excessive inflammatory response elicited by LPS (Erskine et al., supra; Ziv, supra). Current antibiotics approved for internal (i.e., not topical) use only target the pathogen, whereas, BPI has the ability to target both the pathogen and LPS. Both human BPI and its peptide derivatives improve survival during Gram-negative infection and endotoxic shock (Kelly and Cech, supra; Kohn et al., supra; Rogy et al.; supra; Jiang et al., supra; Ammons et al. 1994. J. Infect. Dis. 170: 1473-1482). A peptide of the invention, the peptide corresponding to amino acids 90-99 and 148-161 of bBPI, contains both bactericidal and LPS neutralizing activity, is active in serum, and has therapeutic potential for managing Gram-negative infections in cows and other food production animals, such as goats, sheep, and pigs.

According to the preferred embodiment of the invention, the antimicrobial functional domains of bBPI is the synthetic peptide with sequences corresponding to the combination of amino acids 90-99 and 148-161 (bBPI_{90-99,148-161}) of bBPI.

The antimicrobial protein of the invention is a novel antimicrobial protein that is effective against Gram-negative bacteria, including several mastitis-causing pathogens, e.g., Escherichia coli and Klebsiella, Serratia, Enterobacter, and Pseudomonas genera.

Furthermore, the peptides of the invention may be generated and/or isolated by any means known in the art, including the use of recombinant DNA technologies. For example, U.S. Pat. No. 5,028,530 and U.S. Pat. No. 5,206,154, all of which are hereby incorporated by reference, disclose novel methods for the recombinant production of polypeptides, including antimicrobial peptides. Additional procedures for recombinant production of antimicrobial peptides in bacteria have been described by Piers et al. (1993. Gene 134: 7-13) and U.S. Pat. No. 5,439,807, incorporated by reference, which disclose novel methods for the purification of recombinant BPI expressed in and secreted from genetically-transformed mammalian host cells in culture and discloses how one may produce large quantities of recombinant BPI suitable for incorporation into stable, homogeneous pharmaceutical preparations. The present invention therefore also relates to a strategy of generating a nucleic acid sequence encoding bBPI peptides according to the invention.

The bBPI functional domain peptides of the invention may also be advantageously produced using any such methods. The present invention equally relates to a nucleic acid sequence encoding an above-described bBPI peptide. Those of ordinary skill in the art are able to isolate or chemically synthesize a nucleic acid encoding each of the peptides of the invention. Such nucleic acids are advantageously utilized as components of recombinant expression constructs, wherein the nucleic acids are operably linked with transcriptional and/or translational control elements, whereby such recombinant expression constructs are capable of expressing the peptides of the invention in cultures of prokaryotic, or preferably eukaryotic cells, most preferably mammalian cells, transformed with such recombinant expression constructs.

According to the present invention, “nucleic acid sequence” is to be understood as being a nucleotide sequence which can be of the DNA or the RNA type preferably of the DNA type, especially double-stranded whether it is natural or synthetic origin. This will also include a DNA sequence for which the codons encoding the recombinant peptides according to the invention will have been optimized according to the host organism in which it will be expressed, these optimization methods being well known to those skilled in the art.

As used herein, “recombinant” refers to a nucleic acid molecule which has been obtained by manipulation of genetic material using restriction enzymes, ligases, polymerases and similar genetic engineering techniques as described by, for example, Sambrook et al. 1989. Molecular Cloning: A Laboratory Manual, Second Edition, Cold Spring Harbor Laboratory Press, Cold Spring Harbor, N.Y. or DNA Cloning: A Practical Approach, Vol. I and II (Ed. D. N. Glover), IRL Press, Oxford, 1985. “Recombinant,” as used herein, does not refer to naturally-occurring genetic recombinations.

A “construct” or “chimeric gene construct” refers to a recombinant nucleic acid sequence, generally recombinant DNA, encoding a protein, operably linked to a promoter and/or other regulatory sequences. It is a recombinant nucleic acid sequence that has been generated for the purpose of the expression of a specific nucleotide sequence(s), or is to be used in the construction of other recombinant nucleotide sequences. As used herein, the term “chimeric” refers to two or more DNA molecules which are derived from different sources, strains, or species, which do not recombine under natural conditions, or to two or more DNA molecules from the same species, which are linked in a manner that does not occur in the native genome.

As used herein, the term “express” or “expression” is defined to mean transcription. The regulatory elements are operably linked to the coding sequence of the bBPI gene such that the regulatory element is capable of controlling expression of peptides of bBPI functional domains. “Altered levels” or “altered expression” refers to the production of gene product(s) in transgenic organisms in amounts or proportions that differ from that of normal or non-transformed organisms.

As used herein, the terms “encoding”, “coding”, or “encoded” when used in the context of a specified nucleic acid mean that the nucleic acid comprises the requisite information to guide translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

The term “operably linked” refers to a functional connection between a DNA sequence and a regulatory sequence(s), e.g., a DNA sequence and a regulatory. sequence(s) are connected in such a way as to permit gene expression when the appropriate molecules (e.g., transcriptional activator proteins) are bound to the regulatory sequence(s).

The term “transformation” refers to a permanent or transient genetic change induced in a cell following the incorporation of new DNA (i.e. DNA exogenous to the cell). Where the cell is a mammalian cell, a permanent genetic change is generally achieved by introduction of the DNA into the genome of the cell. When the cell is a bacterial cell, the term usually refers to an extrachromosomal, self-replicating vector which harbors a selectable antibiotic resistance. Extrachromosomal, self replicating vectors harboring selectable antibiotic resistance markers are known for mammalian cells as well; however these are not transferred to progeny cells during cell replication.

The term “cDNA” refers to all nucleic acids that share the arrangement of sequence elements found in native mature mRNA species, where sequence elements are exons and 3′ and 5′ non-coding regions. Normally mRNA species have contiguous exons, with the intervening introns removed by nuclear RNA splicing, to create a continuous open reading frame encoding the protein.

The term “genomic sequence” can refer to a sequence having non-contiguous open reading frames, where introns interrupt the protein coding regions. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a fragment of 100 kb or smaller; and substantially free of flanking chromosomal sequence.

Peptides of the invention may be advantageously synthesized by any of the chemical synthesis techniques known in the art, particularly solid-phase synthesis techniques, for example, using commercially-available automated peptide synthesizers. Such peptides may also be provided in the form of combination peptides, wherein the peptides comprising the combination are linked in a linear fashion one to another and wherein a BPI sequence is present repeatedly in the peptide, with or without separation by “spacer” amino acids allowing for selected conformational presentation. Also provided are branched-chain combinations, wherein the component peptides are covalently linked via functionalities in amino acid sidechains of the amino acids comprising the peptides.

Generally, those skilled in the art will recognize that peptides as described herein may be modified by a variety of chemical techniques to produce compounds having essentially the same activity as the unmodified peptide, and optionally having other desirable properties, such as increased solubility or bioavailability.

Functional domain peptides of this invention can be utilized to generate recombinant multifunctional fusion antimicrobial proteins provided as recombinant hybrid fusion proteins comprising BPI functional domain peptides and at least a portion of at least one other polypeptide. Nucleic acid sequences encoding more than one functional antimicrobial BPI peptide and/or encoding peptides which are specific for other pathogenic bacteria involved in mastitis, i.e., bacteria belonging to more than two different genera, species or substrains can be generated.

Similarly, a nucleic acid sequence encoding a bBPI peptide according to the invention is an encoding sequence which allows disease resistance to be imparted to the organism. It is well understood that this sequence can also be used in combination with another sequence, or sequences, encoding one or more disease resistant properties.

The antimicrobial protein of the invention has been designed to specifically attack Gram-negative mastitis-causing pathogens including, Escherichia coli and other common Gram-negative isolates: Klebsiella, Serratia, Enterobacter, and Pseudomonas genera. Thus, in treating mastitis, several pathogens could be treated with the peptides of the invention.

The mastitis control compositions of the invention comprise the antimicrobial composition of the invention dissolved or suspended in an aqueous carrier or medium. The composition may further generally comprise an acidulant or admixture, a rheology modifier or admixture, a film-forming agent or admixture, a buffer system, a hydrotrope or admixture, an emollient or admixture, a surfactant or surfactant admixture, a chromophore or colorant, and optional adjuvants. The preferred compositions of this invention comprise ingredients which are generally regarded as safe, and are not of themselves or in admixture incompatible with serum or whey. Likewise, ingredients may be selected for any given composition which are cooperative in their combined effects whether incorporated for antimicrobial efficacy, physical integrity of the formulation or to facilitate healing and the health of the teat. Generally, the composition comprises a carrier which functions to dilute the active ingredients and facilitates application to the intended surface. The carrier is generally an aqueous medium such as water, or an organic liquid such as an oil, a surfactant, an alcohol, an ester, an ether, or an organic or aqueous mixture of any of these. Water is preferred as a carrier or diluent in compositions of this invention because of its universal availability and unquestionable economic advantages over other liquid diluents.

### EXAMPLES

Having now generally described this invention, the same will be better understood by reference to certain specific examples, which are included herein only to further illustrate the invention and are not intended to limit the scope of the invention as defined by the claims.

### Example 1

### Bovine BPI Peptides

Peptides corresponding to the sequence of bBPI amino acids 65-99 (bBPI65-99; NSQIRPLPDKGLDLSIRDASIKIRGKWKARKNFIK; SEQ ID NO:2), 142-169 (bBPI142-169; VRIHISGSSLG WLIQLFRKRIESLLQKS; SEQ ID NO:3), or the combination of amino acids 90-99 and 148-161 (bBPI90-99,148-161; KWKARKNFIKGSSLGWLIQL FRKR; SEQ ID NO:4) were commercially synthesized (Genemed Synthesis, Inc., South San Francisco, Calif.). The amino acid numbers correspond with those of the mature form of bBPI. A control peptide (amino acid sequence: MCHWAGGASNTGDARGDV FGKQAG; SEQ ID NO:5) was provided by the same company that synthesized the bBPI-derived peptides. The peptides were reconstituted in citrate-buffered saline (CBS; 20 mM sodium citrate, 150 mM sodium chloride, 0.1% pluoroconic acid, and 0.002% polysorbate 830, pH 5.0; Sigma Chemical Co., St. Louis, Mo.) to a final concentration of 10 mg/ml, aliquotted, and stored at −20° C. All aliquots were thawed only once. Immediately prior to use, aliquotted peptides were diluted to a two-fold higher concentration than the highest concentration used in any given assay.

### Example 2

### Bacterial Strains

Bacterial isolates of Escherichia coli, Klebsiella pneumonia, Serratia marcescens, Pseudomonas aeruginosa, Enterobacter cloacae, and Enterobacter aerogenes, which were obtained from clinical cases of bovine mastitis, were used to assess the bactericidal activity of peptides in a broth microdilution assay. Isolates were obtained from repositories at Cornell University (gift of Dr. Y. H. Schukken; Quality Milk Production Services Program, Cornell University, Ithaca N.Y.) and the USDA Beltsville Agricultural Research Center. E. coli strain P4 (gift of Dr. A. J. Bramley, Institute for Animal Health, Compton Laboratory, Newbury, England), which was originally isolated from a clinical case of mastitis (Bramley, A. J. 1976. J. Dairy Res. 43: 205-211) was also used to assess the bactericidal activity of the peptides in biological fluids. Reference strains of E. coli (ATCC 25922) and Staphylococcus aureus (ATCC 29213) were obtained from a commercial source (American Type Culture Collection (ATCC), Manassas, Va.). To obtain fresh inoculums for evaluation, a sterile loop of each bacterial glycerol stock was streaked on a blood agar plate. After an overnight incubation at 35° C., a single uniform colony was picked and sub-passaged by streaking on a new blood agar plate and incubating the plate overnight at 35° C.

### Example 3

### Bacteriostatic and Bactericidal Activity of Bovine BPI Peptides Against Clinical Mastitis Bacterial Isolates

To evaluate the antimicrobial activity of the peptides against various clinical mastitis bacterial isolates, the minimum inhibitory concentration (MIC) and the minimum bactericidal concentration (MBC) of these peptides against the isolates were determined using a standardized broth microdilution assay. In addition to the mastitis isolates, E. coli (ATCC 25922) and S. aureus (ATCC 29213) reference strains were evaluated. Tetracycline (Sigma Chemical Co.) MIC and MBC values for the various bacteria were determined as well. The assays were performed in accordance with Clinical and Laboratory Standards Institute (CLSI) [formerly National Committee for Clinical Laboratory Standards (NCCLS)] guidelines (National Committee for Clinical Laboratory Standards. 1999. Methods for Determining Bacterial Activity of Antimicrobial Agents: Approved Guideline. Wayne, Pa.; National Committee for Clinical Laboratory Standards. 2002. Performance Standards for Antimicrobial Disk and Dilution Susceptibility Tests for Bacteria Isolated from Animals: Approved Standard. 2^{nd} Edition, Wayne, Pa.). First, a single colony of each fresh bacterial inoculum was diluted in cation-adjusted Mueller-Hinton broth (BD Biosciences, Franklin Lakes, N.J.) and the suspension adjusted to achieve a transmittance equivalent to a 0.5 McFarland standard (10⁸CFU/ml) using a calorimeter. The bacterial suspension was then diluted 100-fold in cation-adjusted Mueller-Hinton broth to reach a final inoculum concentration of 10⁶ CFU/ml. Fifty microliters of the inoculum was added to the wells of a 96-well round bottom plate containing 50 μl of either broth alone, or broth containing serially-diluted peptide or tetracycline. A negative control column was included in the plate that included 100 μl of broth alone. The plates were incubated for 18 h at 35° C. with ambient air circulation and without shaking. The wells were subsequently inspected for cloudiness and pellet formation using a magnifying glass plate reader. The lowest concentration at which no pellet or cloudiness was apparent in a given well was identified as the MIC. The MBC values were determined by plating 10 μl aliquots from the 96-well plates used to determine MIC values onto blood agar plates. The plates were incubated overnight at 35° C. and colonies subsequently enumerated. The MBC values were determined as the lowest concentration at which ≧99.9% of inoculated bacteria were killed. All assays were performed in triplicate and the MIC and MBC range reported.

The spectrum of activity of three bBPI peptides against various Gram-negative bacteria isolated from clinical cases of mastitis was assessed using the broth microdilution susceptibility assay (Table 1). The range of MIC and MBC values from three independent assays are shown.

TABLE 1
Antimicrobial activity of bBPI peptides against clinical mastitis isolates of
Gram-negative bacteria.

| Gram-neg | bBPI₆₅₋₉₉ | | bBPI₁₄₂₋₁₆₉ | | bBPI_{90-99,148-161} | | Tetracycline | |
|---|---|---|---|---|---|---|---|---|
| Bacteria | MIC | MBC | MIC | MBC | MIC | MBC | MIC | MBC |
| Enterobacter | | | | | | | | |
| aerogenes | | | | | | | | |
| Isolate #1 | >256 | >256 | 256->256 | >256 | 64 | 64 | 128 | >128 |
| Enterobacter | | | | | | | | |
| cloacae | | | | | | | | |
| Isolate #1 | >256 | >256 | >256 | >256 | 32 | 64 | 128 | >128 |
| Isolate #2 | >256 | >256 | >256 | >256 | 32 | 32-64 | 4 | >128 |
| Isolate #3 | >256 | >256 | >256 | >256 | 64 | 64 | 128 | >128 |
| Isolate #4 | >256 | >256 | 256->256 | 256->256 | 64 | 64 | 4 | >128 |
| Escherichia | | | | | | | | |
| coli | | | | | | | | |
| Isolate #1 | >256 | >256 | 256 | 256 | 16 | 16-32 | 4 | 128 |
| Isolate #2 | >256 | >256 | 256 | 256 | 32-64 | 32-64 | 4 | >128 |
| Isolate #3 | >256 | >256 | 128-256 | 128-256 | 32-64 | 32-64 | 4 | >128 |
| Isolate #4 | >256 | >256 | 128-256 | 128-256 | 32-64 | 64 | 4 | >128 |
| Isolate #5 | >256 | >256 | 256 | 256 | 64 | 64 | 4 | 128 |
| Strain P4 | >256 | >256 | 128-256 | 128-256 | 16-64 | 32-64 | 1 | >128 |
| ATCC 25922 | >256 | >256 | 128-256 | 128-256 | 32-64 | 64 | 0.5-1 | 64 |
| Klebsiella | | | | | | | | |
| pneumoniae | | | | | | | | |
| Isolate #1 | >256 | >256 | >256 | >256 | 32-64 | 32-64 | 64 | >128 |
| Isolate #2 | >256 | >256 | >256 | >256 | 32-64 | 32-64 | 128 | >128 |
| Isolate #3 | >256 | >256 | >256 | >256 | 32-64 | 32-64 | 1 | >128 |
| Isolate #4 | >256 | >256 | 256 | 256 | 32-64 | 32-64 | 64 | >128 |
| Isolate #5 | >256 | >256 | >256 | >256 | 32-64 | 32-64 | 16 | >128 |
| Isolate #6 | >256 | >256 | >256 | >256 | 64 | 64 | 0.5 | 128 |
| Pseudomonas | | | | | | | | |
| aeruginosa | | | | | | | | |
| Isolate #1 | >256 | >256 | >256 | >256 | 128 | 128 | 32 | >128 |
| Isolate #2 | >256 | >256 | >256 | >256 | 128 | 128 | 16 | >128 |
| Isolate #3 | >256 | >256 | >256 | >256 | 64 | 64 | 32 | 128 |
| Isolate #4 | >256 | >256 | >256 | >256 | 128 | 128 | 16 | >128 |
| Isolate #5 | >256 | >256 | >256 | >256 | 128 | 128 | 32 | >128 |
| Isolate #6 | >256 | >256 | >256 | >256 | 128 | 128 | 16 | >128 |
| Serratia | | | | | | | | |
| marcescens | | | | | | | | |
| Isolate #1 | >512 | >512 | >512 | >512 | >512 | >512 | 32 | >128 |
| Isolate #2 | >512 | >512 | >512 | >512 | >512 | >512 | 64 | >128 |
| Isolate #3 | >512 | >512 | >512 | >512 | >512 | >512 | 64 | >128 |
| Isolate #4 | >512 | >512 | >512 | >512 | >512 | >512 | 64 | >128 |
| Isolate #5 | >512 | >512 | >512 | >512 | >512 | >512 | 64 | >128 |
| Isolate #6 | >512 | >512 | >512 | >512 | >512 | >512 | 64 | >128 |
| Staphylococcus | | | | | | | | |
| aureus | | | | | | | | |
| ATCC 29213 | >256 | >256 | 128-256 | 128-256 | 64 | 64-128 | <0.25-0.5 | 128->128 |

Minimum inhibitory (MIC) and bactericidal (MBC) concentrations are reported as μg/ml. The range of values from three broth microdilution assays evaluating peptide antimicrobial activity is shown.

At the concentrations tested, bBPI₆₅₋₉₉ demonstrated no growth-inhibitory or bactericidal activity against any of the bacterial isolates. A limited spectrum of isolates were susceptible to bBPI₁₄₂₋₁₆₉ at concentrations ≧128 μg/ml. Within the range of concentrations tested, all strains of E. coli were susceptible, whereas only one isolate of K. pneumoniae and E. cloacae demonstrated repeatable susceptibility to bBPI₁₄₂₋₁₆₉ in all three independent experiments. The third peptide, bBPI_{90-99,148-161}, displayed the lowest MIC and MBC values against the widest spectrum of bacteria. With the exception of S. marcescens, bBPI_{90-99,148-161} had both growth-inhibitory activity on, and bactericidal activity against, all bacteria tested. The peptide's MIC and MBC values were all ≦64 μg/ml for all isolates of E. coli, K. pneumoniae, and Enterobacter spp., and ≦128 μg/ml for all P. aeruginosa isolates. Initial assays of susceptibility of S. marcescens revealed that it was refractory to the antimicrobial effects of any of the BPI peptides tested at concentrations ≦256 μg/ml. Thus, the dose-response range of the peptides was increased so as to assay the efficacy of these peptides up to a concentration of 512 μg/ml. Even at the higher concentration assayed, none of the peptides demonstrated any ability to inhibit S. marcescens growth. In addition to the Gram-negative isolates, bBPI₁₄₂₋₁₆₉ and bBPI_{90-99,148-161} exhibited growth-inhibitory and bactericidal activity against the quality control reference strain of S. aureus.

Each bacterial isolate was also evaluated for its susceptibility to tetracycline (Table 1) to enable comparison with the MIC and MBC values determined for the bBPI peptides. Tetracycline inhibited the growth of all isolates. The tetracycline MIC values for the two reference strains, E. coli ATCC 25922 and S. aureus ATCC 29213, were within the established quality control ranges of 0.5-2 and 0.12-1 μg/ml, respectively (NCCLS, 1999). With the exception of three isolates of Enterobacter spp. and one isolate of K. pneumoniae, the MIC values of tetracycline were lower than that of the most efficacious bBPI peptide, bBPI_{90-99,148-161}. In contrast to the MIC values, the MBC values of tetracycline were almost universally higher than those of bBPI_{90-99,148-161}. The MBC values for the BPI peptide were all equivalent to or only 1-fold higher than corresponding MIC values, whereas, the fold-difference in tetracycline MBC values relative to those of its MIC tended to be higher.

### Example 4

### Bactericidal Activity of Bovine BPI Peptides in Serum, Milk or Whey

Milk and blood samples were aseptically collected from 8 clinically healthy primiparous Holstein cows in mid-lactation with milk somatic cell counts of <150,000 cells/ml. All cows were determined to be free of intramammary pathogens by the absence of growth on blood agar plates spread with milk samples aseptically collected from each animal on 3 different days. For the preparation of whey, milk samples were centrifuged at 44,000×g at 4° C. for 30 min, and the fat layer removed with a spatula. The skim milk was transferred into another sterile tube, centrifuged for an additional 30 min, and the translucent supernatant collected. Milk and whey samples were pasteurized by heating at 63° C. for 30 min. For the preparation of serum, blood was collected from the coccygeal vein into vacutainer tubes containing gel and clot activator (BD Biosciences) for serum separation. The tubes were inverted ×5, the blood allowed to clot for 30 min, and the tubes centrifuged at 1500×g for 15 min. The clear serum supernatants were aseptically transferred into sterile tubes and heat-inactivated by incubating at 56° C. for 30 min. An aliquot of each sample was plated on blood agar plates and samples that were free of detectable bacterial growth were stored at −20° C.

Overnight bacterial cultures of E. coli strain P4 grown in trypticase soy broth were diluted 1:1,000 in brain heart infusion broth and incubated at 37° C. for 2 hours while shaking at 225 rpm. The log-phase bacteria were diluted 1:10 in brain heart infusion broth to obtain a final concentration of 1×10⁶ CFU/ml. 0.01 ml of the bacterial inoculum was added to a 1.5 ml sterile microcentrifuge tube containing 0.04 ml of serum, milk, or whey. A 0.05 ml aliquot of either peptide or CBS alone was added to each tube. The samples were then shaken at 100 rpm for 6 hours at 37° C. The sample mixtures were subsequently serially diluted in sterile phosphate-buffered saline and plated on MacConkey agar plates (BD Biosciences). The plates were incubated overnight at 37° C. and the colonies enumerated.

To determine whether bBPI₁₄₂₋₁₆₉ and bBPI_{90-99,148-161} retained their bactericidal activity in physiological fluids, the peptides were incubated with E. coli strain P4 (1×10⁵ CFU) in the presence of serum (Table 2) or milk (Table 3). Because bBPI₆₅₋₉₉ was shown to have no bactericidal activity in broth, this peptide was assayed in parallel as a negative control. In the presence of serum, bBPI_{90-99,148-161} demonstrated bactericidal activity against E. coli at concentrations ≧10 μg/ml (Table 2). In contrast, neither bBPI₆₅₋₉₉ nor bBPI₁₄₂₋₁₆₉ demonstrated bactericidal activity at concentrations ≦100 μg/ml in serum.

TABLE 2
Antimicrobial activity of bBPI peptides against E. coli strain
P4 in serum.

| Concentration | | bBPI₆₅₋₉₉ | | bBPI₁₄₂₋₁₆₉ | | bBPI_{90-99,148-161} | |
|---|---|---|---|---|---|---|---|
| (μg/ml) | n | Mean | S.E. | Mean | S.E. | Mean | S.E. |
| 0 | 8 | 8.40 | 0.05 | 8.40 | 0.05 | 8.40 | 0.05 |
| 0.1 | 8 | 8.27 | 0.03 | 8.42 | 0.05 | 8.25 | 0.03 |
| 1 | 8 | 8.28 | 0.04 | 8.38 | 0.03 | 8.15 | 0.06 |
| 10 | 8 | 8.23 | 0.03 | 8.29 | 0.04 | 0 | 0 |
| 100 | 8 | 8.07 | 0.05 | 7.96 | 0.07 | 0 | 0 |

Mean (±S.E.) log₁₀ colony forming units are reported.
n = number of replicates

TABLE 3
Antimicrobial activity of bBPI peptides against E. coli strain
P4 in milk.

| Concentration | | bBPI₆₅₋₉₉ | | bBPI₁₄₂₋₁₆₉ | | bBPI_{90-99,148-161} | |
|---|---|---|---|---|---|---|---|
| (μg/ml) | n | Mean | S.E. | Mean | S.E. | Mean | S.E. |
| 0 | 8 | 8.69 | 0.05 | 8.69 | 0.05 | 8.69 | 0.05 |
| 20 | 8 | 8.69 | 0.06 | 8.67 | 0.03 | 8.65 | 0.08 |
| 200 | 8 | 8.54 | 0.10 | 8.55 | 0.07 | 7.89 | 0.12 |
| 2000 | 8 | 8.06 | 0.12 | 7.99 | 0.07 | 6.55 | 0.27 |

Mean (±S.E.) log₁₀ colony forming units are reported.
n = number of replicates

Initial evaluation of the peptides in milk revealed no bactericidal activity against E. coli (data not shown); therefore, a more extended dose-response range was used to evaluate higher concentrations of the peptides (Table 3). Even at concentrations up to 2 mg/ml, none of the peptides demonstrated bactericidal activity against E. coli in milk. However, bBPI_{90-99,148-161} was able to inhibit >99% of the growth of E. coli in milk. Because milk appeared to have an inhibitory effect on the bactericidal activity of bBPI_{90-99,148-161}, the peptides were subsequently evaluated for their ability to retain activity in whey, which is the casein-depleted, protein-rich liquid fraction of milk (Table 4). In the presence of whey, bBIP_{90-99,148-161} displayed bactericidal activity against E. coli at concentrations ≧10 μg/ml. In contrast, neither bBPI₆₅₋₉₉ nor bBPI₁₄₂₋₁₆₉ demonstrated bactericidal activity at any concentration tested in whey.

TABLE 4
Antimicrobial activity of bBPI peptides against E. coli strain
P4 in whey.

| Concentration | | bBPI₆₅₋₉₉ | | bBPI₁₄₂₋₁₆₉ | | bBPI_{90-99,148-161} | |
|---|---|---|---|---|---|---|---|
| (μg/ml) | n | Mean | S.E. | Mean | S.E. | Mean | S.E. |
| 0 | 8 | 8.28 | 0.24 | 8.32 | 0.23 | 8.27 | 0.05 |
| 0.1 | 8 | 8.47 | 0.14 | 8.29 | 0.18 | 8.17 | 0.03 |
| 1 | 8 | 8.26 | 0.18 | 8.44 | 0.14 | 7.97 | 0.04 |
| 10 | 8 | 8.16 | 0.16 | 8.10 | 0.20 | 0 | 0 |
| 100 | 8 | 7.36 | 0.30 | 7.45 | 0.21 | 0 | 0 |

Mean (±S.E.) log₁₀ colony forming units are reported.
n = number of replicates

### Example 5

### LPS Neutralizing Activity of Bovine BPI Peptides

The ability of the peptides to neutralize LPS was determined using a commercially available Limulus amebocyte lysate (LAL) assay (Cambrex BioScience Walkersville, Inc., Walkersville, Md.). As a positive control, polymyxin B (Sigma Chemical Co.) was evaluated in parallel with the peptides. Increasing concentrations of peptides or polymyxin B were incubated with 1 ng of highly purified bacterial LPS (Sigma Chemical Co.), derived from E. coli 0111:B4, in a 500 μl reaction volume of endotoxin-free water for 30 min at 37° C. while shaking at 100 rpm. Following the incubation, the amount of free LPS was determined in each sample according to the manufacturer's instructions. Briefly, 50 μl of the above mixture was placed into a 96-well microtiter plate and the reaction initiated by the addition of an equal volume of amoebocyte lysate. Following an incubation at 37° C. for 10 min, 100 μl of chromogenic substrate was added and the plate incubated for 5-10 min at 37° C. The reaction was stopped by the addition of 50 μl of glacial acetic acid and the absorbance measured at a wavelength of 405 nm on a microplate reader (Biotec Instruments, Inc., Winooski, Vt.). The amount of non-bound LPS was extrapolated from a standard curve and the percent inhibition calculated according to the following formula:

$$\frac{[ \begin{matrix}( amount of free LPS in control samples ) - \\ ( amount of free LPS in test samples )\end{matrix} ] \times 100}{( amount of free LPS in control samples )}$$

To determine whether the bBPI peptides were able to neutralize LPS, increasing concentrations of the peptides were incubated with LPS (1 ng) and the resulting mixture assayed with the LAL assay (FIG. 1). As a positive and negative control, respectively, polymyxin B and a non-BPI-derived peptide were also assayed. At the lowest concentration tested (1 μg/ml), bBPI₆₅₋₉₉ and bBPI₁₄₂₋₁₆₉ inhibited approximately 20% of LPS activity, whereas, bBPI_{90-99,148-161} displayed no inhibition. Within the dose-response range tested, bBPI₆₅₋₉₉ maximally inhibited 50% of LPS activity. In contrast, bBPI₁₄₂₋₁₆₉ and bBPI_{90-99,148-161} were able to neutralize ≧93% of LPS activity at peptide concentrations of 30 and 100 μg/ml, respectively. Polymyxin B inhibited LPS activity in dose-dependent manner, whereas the control peptide demonstrated no inhibitory effect even at the highest concentration assayed (100 μg/ml).

All publications and patents mentioned in this specification are herein incorporated by reference to the same extent as if each individual publication or patent was specifically and individually indicated to be incorporated by reference.

The foregoing description and certain representative embodiments and details of the invention have been presented for purposes of illustration and description of the invention. It is not intended to be exhaustive or to limit the invention to the precise forms disclosed. It will be apparent to practitioners skilled in this art that modifications and variations may be made therein without departing from the scope of the invention.

We claim:

1. A peptide which is an amino acid sequence of bovine bactericidal permeability increasing protein (BPI; SEQ ID NO:1) from about position 65 to about position 99 of mature bovine BPI (bBPI), subsequences thereof and variants of the sequence or subsequence thereof, having a biological activity that is an activity of bBPI wherein said activity is LPS neutralizing activity and said subsequences having an amino acid sequence different from human BPI.

2. The peptide of claim 1 having the amino acid sequence NSQIRPLPDKGLDLSIRDAS IKIRGKWKARKNFIK identified by SEQ ID NO:2.

3. A peptide which contains two or three of the same or different peptides according to claim 1 covalently linked together.

4. A pharmaceutical composition comprising a peptide according to claim 1 and a pharmaceutically-acceptable carrier or diluent.

5. A peptide which is an amino acid sequence of bovine bactericidal permeability increasing protein (bBPI; SEQ ID NO:1) from about position 142 to about position 169 of mature bBPI, subsequences thereof and variants of the sequence or subsequence thereof, having a biological activity that is an activity of bBPI wherein said activity is LPS neutralizing activity and said subsequences having an amino acid sequence different from human BPI.

6. The peptide of claim 5 having the amino acid sequence VRIHISGSSLGWLIQL FRKRIESLLQKS identified by SEQ ID NO:3.

7. A peptide which contains two or three of the same or different peptides according to claim 5 covalently linked together.

8. A pharmaceutical composition comprising a peptide according to claim 5 and a pharmaceutically-acceptable carrier or diluent.

9. A peptide which is an amino acid sequence of bBPI from about position 90 to about position 99 covalently linked together to a peptide which is an amino acid sequence of mature bBPI from about position 148 to about position 161 of SEQ ID NO:1, subsequences thereof and variants of the sequence or subsequence thereof, having a biological activity that is an activity of BPI wherein said activity is bacteriostatic activity, bactericidal activity and LPS neutralizing activity and said subsequences having an amino acid sequence different from human BPI.

10. The peptide of claim 9 having the amino acid sequence KWKARKNFIKGS SLGWLIQLFRKR identified by SEQ ID NO:4).

11. A pharmaceutical composition comprising a peptide according to claim 9 and a pharmaceutically-acceptable carrier or diluent.

12. A nucleic acid encoding the peptide of any one of claims 1, 5, and 9.

13. A chimeric gene comprising in operable linkage the nucleic acid of claim 1 and regulatory elements functional in a host organism, wherein the regulatory elements comprise a promoter from a gene that is expressed in the host organism.

14. A cloning vector comprising the chimeric gene of claim 13.

15. An expression vector comprising the chimeric gene of claim 13.

16. A process for transforming a host cell, comprising stably integrating the nucleic acid of claim 1 or the chimeric gene of claim 13 into the host cell.

17. An isolated host cell transformed with the nucleic acid according to claim 1 or the chimeric gene according to claim 13.

18. A method for killing gram-negative bacteria comprising contacting the bacteria with an effective amount of a peptide which has an amino acid sequence of bBPI from about position 65 to about position 99 of SEQ ID NO: 1, subsequences thereof and variants of the sequence or subsequence thereof, having a biological activity that is an activity of BPI wherein said activity is LPS neutralization and said subsequences having an amino acid sequence different from human BPI.

19. A method for killing gram-negative bacteria comprising contacting the bacteria with an effective amount of a peptide which has an amino acid sequence of bBPI from about position 142 to about position 169 of SEQ ID NO: 1, subsequences thereof and variants of the sequence or subsequence thereof, having a biological activity that is an activity of BPI wherein said activity is LPS neutralization and said subsequences having an amino acid sequence different from human BPI.

20. A method for killing gram-negative bacteria comprising contacting the bacteria with an effective amount of a peptide which has an amino acid sequence of bovine BPI from about position 90 to about position 99 covalently linked together to a peptide which is an amino acid sequence of mature bBPI from about position 148 to about position 161 of SEQ ID NO:1, subsequences thereof and variants of the sequence or subsequence thereof, having a biological activity that is an activity of BPI wherein said activity is LPS neutralization, bactericidal activity, and bacteriostatic activity and said subsequences having an amino acid sequence different from human BPI.

21. A method for killing gram-negative bacteria comprising contacting the bacteria with an effective amount of a peptide in which two or three of the same or different peptides according to claim 18, 19, or 20 are directly covalently linked together, having a biological activity that is an activity of BPI wherein said activity is LPS neutralization, bactericidal activity, and bacteriostatic activity

22. A method according to claim 18 wherein the peptide has the amino acid sequence: NSQIRPLPDKGLDLSIRDASIKIRGKWKARKNFIK identified by SEQ ID NO:2

23. A method according to claim 19 wherein the peptide has the amino acid sequence: VRIHISGSSLGWLIQLFRKRIESLLQKS identified by SEQ ID NO:3.

24. A method according to claim 20 wherein the peptide has the amino acid sequence: KWKARKNFIKGSSLGWLIQLFRKR identified by SEQ ID NO:4).
